(19) United States
(12) Patent Application Publication
(10) Pub. No.: US 2026/0000727 A1
(43) Pub. Date: Jan. 1, 2026

(54) METHOD FOR VALORIZATION OF RICE HUSK

(71) Applicants: Nasser Awwad, Abha (SA); Mohamed Hamdy, Abha (SA); Hisham Abd-Rabboh, Abha (SA); Khalid Fawy, Abha (SA); Serag Eldin I. Elbehairi, Abha (SA); Ali A. Alshati, Abha (SA); Mohammad Y. Alfaifi, Abha (SA); Hala A. Ibrahium, Abha (SA); Saad Alamri, Abha (SA)

(72) Inventors: Nasser Awwad, Abha (SA); Mohamed Hamdy, Abha (SA); Hisham Abd-Rabboh, Abha (SA); Khalid Fawy, Abha (SA); Serag Eldin I. Elbehairi, Abha (SA); Ali A. Alshati, Abha (SA); Mohammad Y. Alfaifi, Abha (SA); Hala A. Ibrahium, Abha (SA); Saad Alamri, Abha (SA)

(21) Appl. No.: 18/754,426

(22) Filed: Jun. 26, 2024

(51) Int. Cl. A61K 36/899; A61K 33/00; A61K 33/38; A61K 33/44; A61K 47/69

(52) U.S. Cl. CPC A61K 36/899; A61K 33/00; A61K 33/38; A61K 33/44; A61K 47/6929

(57) ABSTRACT

A process to treat the rice husk and straw by using chemical agents to obtain valuable products with industrial impact. The source of the rice husk and straw is the residuals of Hassawi rice. Focus is on integrated large-scale production of silica that is separated from the one-pot continuous process as raw materials. It also feeds the paper industry with a massive volume of pulp produced after silica extraction for value-added materials. The process includes alkaline treatment of washed husk and straw after shredding to increase the applied pH to certain values. The obtained products are mesoporous silica, activated carbon, graphene oxide, lignocelluloses, pulp, lignin-free cellulose. The obtained materials can serve in the production of glass, ceramics, catalysts, super capacitors, bio-fuel, salt-free water, anti-cancer agents, drug delivery and printing paper. The obtained products can be applied in water treatment and in pharmaceutical and biomedical applications.

### COPYRIGHT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### BACKGROUND OF THE INVENTION

### 1) Field of the Invention

The invention relates to the field of recyclables, and more specifically to a process using a silica extraction technique to convert Hassawi rice husk and straw waste into several valuable products.

### 2) Description of Related Art

There is an enormous amount of biomass byproducts produced from the Hassawi rice plant (e.g. husk and straw). This is found exclusively in Saudi Arabia. This waste is currently burned in the fields and creates a considerable amount of pollution, which has seriously negative effect on the environment and health.

Currently, the rice husk or straw is burned in the fields, which causes environmental and health hazards. What is needed is an integrated process for the elimination of harmful effects of the burning and reducing associated costs while converting Hassawi rice husk and straw waste into several valuable products.

### SUMMARY OF THE INVENTION

The instant invention in one form is directed to a chemical treatment method for the recycling of Hassawi rice husk and straw agricultural waste gathered from Al-Ahsa region at western Saudi Arabia, in order to produce several natural important materials including silica, lignocelluloses, and activated carbon.

The conversion process consists of several consecutive chemical treatment in order to obtain the bio-based materials. In a typical conversion process, the collected rice straw/husk is shredded into small pieces (from 3-7 cm length). Then, the shredded pieces are washed by water to remove dust, and dried at room temperature overnight. The first treatment step is carried out by using alkaline solution, in which the clean straw/husk is immersed in a pH between 9 to 10. For the extraction of silica from rice husk and straw by potassium hydroxide, raw rice straw is shredded into 2-cm pieces using plastic scissors. Afterwards, both rice husk and shredded rice straw are stirred separately in 5% KOH solution at 1:12 (g/ml) weight ratio of solid:solution, respectively. Then, the mixtures are heated to boiling for 30 min, then left overnight, filtered, and washed twice with distilled water until filtrates becomes neutral. The filtrates are collected, and subsequently used, as they are predicted to contain leached silica, which is the major component of the ash. To the filtrates, 10% HCl is added, until the pH of the solutions reached (5-7), to precipitate dissolved silica. The formed precipitates are filtered, dried and weighed. Parts of the obtained silica are burnet in an electric muffle furnace at 500, 700, and 900° C. for 1.5 h. Silica is then functionalized for water desalination studies. The formed precipitate (lignin) is collected, washed and dried. Then, the pH is increased to reach 14 until silicate-rich filtrate is obtained. The pH of the silicate rich filtrate is reduced to 7 by using mineral acid to obtained silicate solution. The obtained silicate solution reacted with Pluronic 127 to obtain a dense phase, which upon calcination at 600° C. produced mesoporous silica with a distinguished sphere particle shape. On the other hand, the pH of the obtained residual is reduced again to reach 1 to obtained lignocellulose as a precipitate. The lignocellulose is dried and carbonized at elevated temperature to obtained graphite. The formed graphite is converted to graphene oxide by using acidic treatment at 0° C. Moreover, the silicate-free residual is washed to remove any alkaline contaminations to obtained silicate-free pulp. The silicate-free pulp is carbonized to obtain activated carbon. Therefore, starting from Hassawi rice straw/husk, bio-based products such as lignin, silicate, mesoporous silica, graphite, graphene oxide, lignocellulose, activated carbon are obtained by chemical treatment.

These promising low-cost and environmental friendly natural materials can be applied in the fields of bio-fuel production, water desalination, energy storage, and anticancer medications and studies. In order to maximize the use of rice husk, future research should focus on integrated large-scale production of silica that is separated from the one-pot continuous process as raw materials. It should also feed the paper industry with a massive volume of pulp produced after silica extraction for value-added materials.

These and other objects, features, and advantages of the present invention will become more readily apparent from the attached drawings and the detailed description of the preferred embodiments, which follow.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 is a flow diagram showing the extraction of silica from rice husk and straw by potassium hydroxide; and

FIG. 2 is a flow diagram showing an alkaline treatment of rice husk and straw.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The term valorization as used in the specification is meant to mean the act of making something valuable or useful from an existing substance as defined in the Cambridge Dictionary.

As stated above, there is an enormous, huge amount of biomass byproducts produced from the Hassawi rice plant (e.g. husk and straw). This is found exclusively in Saudi Arabia. This waste is currently burned in the fields and creates a considerable amount of pollution, which has seriously negative effect on the environment and health. The invention claimed here solves this problem.

The invention delivers a process to use Hassawi rice waste to produce several valuable products through chemical treatment. This is accomplished via the extraction of silica from rice husk and straw by potassium hydroxide The produced products include mesoporous silica, graphite, graphene oxide, lignocellulose, cellulose, and bioethanol. The invention demonstrates the application of these produced products in the fields of catalysis, biofuel production, super capacitor production, drug delivery, anti-cancer compounds, paper manufacturing, and water treatment. It provides an environmentally friendly and economically effective method to the current implementation.

The claimed invention differs from what currently exists. The invention introduces an integrated process for the extraction of silica from rice husk and straw by potassium hydroxide. In addition to that, the invention also focuses on the minimization of the pollution which is produced as a result of burning waste in the fields. To the best of our knowledge, this is the first study regarding the conversion of Hassawi agriculture waste into valuable products which can be utilized in several industries.

Also, it can produce several chemical compounds can be obtained from the current invention such as, mesoporous silica, graphite, graphene oxide, lignocellulose, cellulose, and bioethanol.

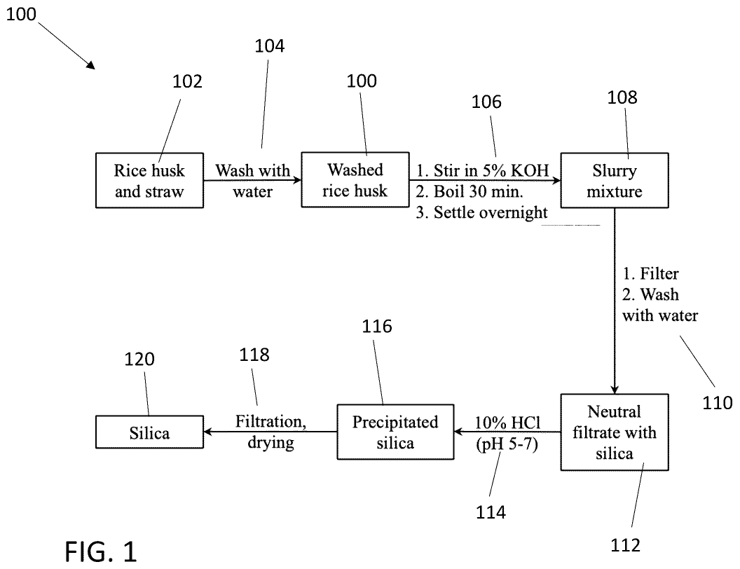

Referring now to FIG. 1, a flow diagram showing a general process or method for the extraction of silica from rice husk and straw by potassium hydroxide is shown. The method includes several consecutive chemical treatment in order to obtain the bio-based materials. In an example conversion process 100, the collected rice husk and straw 102 are shredded into small pieces (from 3-7 cm length). Then, the shredded pieces are washed 104 by water to remove dust, and dried at room temperature overnight. The first treatment step is carried out by using alkaline solution, in which the clean straw/husk is immersed in a pH between 9 to 10. For the extraction of silica from rice husk and straw by potassium hydroxide, raw rice straw is shredded into 2-cm pieces using plastic scissors. Afterwards, both rice husk and shredded rice straw are stirred separately in 5% KOH solution at 1:12 (g/ml) weight ratio of solid:solution, respectively 106. Then, the mixtures 108 are heated to boiling for 30 min, then left overnight, filtered, and washed twice 110 with distilled water until filtrates becomes neutral 112. The filtrates are collected, and subsequently used, as they are predicted to contain leached silica, which is the major component of the ash. To the filtrates, 10% HCl 114 is added, until the pH of the solutions reached (5-7), to precipitate dissolved silica 116. The formed precipitates are filtered, dried and weighed 118. Parts of the obtained silica are burnt in an electric muffle furnace at 500, 700, and 900° C. for 1.5 h. Silica is then functionalized for water desalination studies 120. The formed precipitate (lignin) is collected, washed and dried. Then, the pH is increased to reach 14 until silicate-rich filtrate is obtained. The pH of the silicate rich filtrate is reduced to 7 by using mineral acid to obtained silicate solution. The obtained silicate solution reacted with Pluronic 127 to obtain a dense phase, which upon calcination at 600° C. produced mesoporous silica with a distinguished sphere particle shape. On the other hand, the pH of the obtained residual is reduced again to reach 1 to obtained lignocellulose as a precipitate. The lignocellulose is dried and carbonized at elevated temperature to obtained graphite. The formed graphite is converted to graphene oxide by using acidic treatment at 0° C. Moreover, the silicate-free residual is washed to remove any alkaline contaminations to obtained silicate-free pulp. The silicate-free pulp is carbonized to obtain activated carbon. Therefore, starting from Hassawi rice straw/husk, bio-based products such as lignin, silicate, mesoporous silica, graphite, graphene oxide, lignocellulose, activated carbon are obtained by chemical treatment.

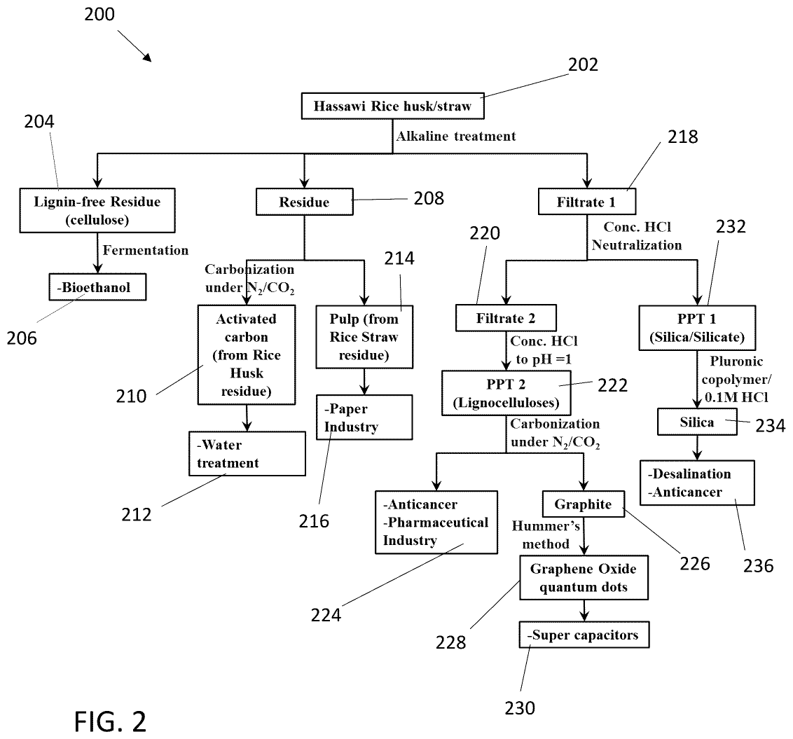

FIG. 2 shows a flow diagram 200 showing detailed valorization of rice husk and straw using alkaline treatment of rice husk and straw. The method includes providing a quantity of rice husks 202, shredding the quantity of rice husks into small husk fragments and washing the small husk fragments to remove dust and waste from the small husk fragments. The method includes forming a mixture by treating the small husk fragments with an alkaline solution until the pH of the mixture is between 9 and 10, forming a lignin-free cellulose 204 and forming a silicate-rich filtrate 208 and a silicate-free residue 218 by increasing the pH of the mixture to about 14. The lignin-free cellulose 204, silicate-rich filtrate 208 and silicate-free residue 218 each have at least one use. The lignin-free cellulose 204 is formed into a bioethanol 206 by fermentation. The silicate-rich filtrate 208 is carbonized under N2 and CO2 to produce activated carbon 210 which may be used in water treatment 212. The silicate-rich filtrate 208 may be formed into a pulp 214 and used in the paper industry 216.

The silicate-free residue 218 is treated with HCL to form a second filtrate 220 a mineral acid to reduce the pH to about 1 to precipitate lignocellulose 222 and dried and ground into fine powder. The method includes carbonizing the lignocellulose 222 at elevated temperature 600-9000 C under N2 and CO2 to obtained graphite 226 and converting the graphite into graphene oxide 228 by adding the graphite into concentrated H2SO4 and NaNO3 at 0° C. The graphene oxide 228 may be used in making super capacitors 230. The carbonized lignocelluloses may also be used in the anticancer industry or the pharmaceutical industry. The silicate-free residue 218 treated with HCL may also be used to form a silica or silicate 232, 234 for desalination or used for anticancer studies or products 236.

The method includes adding stochiometric KMnO4, after dilution, adding H2O2 to obtain graphene oxide and washing the silicate-free residue to remove alkaline contaminations and the silicate-free pulp. The method includes carbonizing the silicate-free pulp at elevated temperature 600-900° C. to obtain activated carbon and functionalizing the bio-based mesoporous silica by doping with nanoparticles of silver wherein a produced material exhibits high cytotoxic activity against three different tumor cell lines of breast (MCF-7), liver (HepG2), and colon (HCT 116) over a concentration range of 0.01 to 1000 g through apoptotic mechanism. The method includes functionalizing the bio-based mesoporous silica by adding amino groups on the surface wherein the obtained material exhibits high cytotoxic activity against three different tumor cell lines of breast (MCF-7), liver (HepG2), and colon (HCT 116) over a concentration range of 0.01 to 1000 g through apoptotic mechanism. The method includes fermenting the lignin-free cellulose by Saccharomyces cerevisiae wherein a bioethanol yield produced from the fermentation is 0.454 ml ethanol/g fermentable.

These promising low-cost and environmentally friendly natural materials can be applied in the fields of bio-fuel production, water desalination, energy storage, and anticancer medications and studies. In order to maximize the use of rice husk, future research should focus on integrated large-scale production of silica that is separated from the one-pot continuous process as raw materials. It should also feed the paper industry with a massive volume of pulp produced after silica extraction for value-added materials.

All the produced materials are originated from one source, which is the Hassawi rice husk/straw. The rice is treated through an alkaline process. This produced three byproducts, cellulose, residue and filtrate. For the extraction of silica process from rice husk and straw by potassium hydroxide, the raw rice straw is shredded into 2-cm pieces using plastic scissors. The byproducts are used to produce end products that can be used successfully in several industrial applications.

The current invention introduces an integrated process to treat the rice husk and straw by using different chemical agents in order to obtain valuable products with industrial impact. The source of the rice husk and straw is the residuals of Hassawi rice, which cultivates in Al-Ahsa Governorate in Saudi Arabia. The main process is the extraction of silica from rice husk and straw by potassium hydroxide The process involves the alkaline treatment of the washed husk and straw after shredding to increase the applied pH to certain values. The obtained products are: mesoporous silica, activated carbon, graphene oxide, lignocellulose, pulp, lignin-free cellulose. The obtained materials can serve in the production of glass, ceramics, catalysts, super capacitors, bio-fuel, salt-free water, anti-cancer agents, drug delivers and paper. Moreover, the obtained products can be applied in water treatment and in the pharmaceutical and biomedical applications.

In one embodiment, the process includes chemical conversion of the husk and straw, production of the bio-based products, and applications of the produced materials. The process includes:
1—Chemical treatment for the husk and straw
2—Physical separation
3—Functionalization of the bio-based products
4—Thermal treatment to obtained the final products
5—The utilization of the produced materials in several industrial application

Preferably, the method is integrated and there are no optional steps. The process can be improved through carrying out from semi-pilot plant into pilot plant.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method for valorization of risk husks and straw, the method comprising:
chemically treating the rice husk and straw;
separating bio-based products of the chemically treated rice husk and straw;
functionalizing at least one of the separated bio-based products;
thermally treating to obtained at least one usable product; and
using the at least one usable product in an industrial application.

2. The method according to claim 1 wherein chemically treating the rice husk and straw includes forming a silicate-rich filtrate and a silicate-free residue by increasing the pH of the mixture to about 14, treating the silicate-rich filtrate by using mineral acid to reduce the pH to about 7 to obtain a silicate solution, and reacting the silicate solution with pluronic 127 copolymer under acidic conditions to obtain a homogeneous gel, which by calcination forms a bio-based mesoporous silica.

3. The method according to claim 1 wherein functionalizing at least one of the separated bio-based products includes functionalizing an obtained bio-based mesoporous silica by doping with nanoparticles of silver wherein a produced material exhibits high cytotoxic activity against three different tumor cell lines of breast (MCF-7), liver (HepG2), and colon (HCT 116) over a concentration range of 0.01 to 1000 g through apoptotic mechanism and functionalizing the bio-based mesoporous silica by adding amino groups on the surface wherein the obtained material is exhibited high cytotoxic activity against three different tumor cell lines of breast (MCF-7), liver (HepG2), and colon (HCT 116) over a concentration range of 0.01 to 1000 g through apoptotic mechanism.

4. The method according to claim 1 wherein thermally treating to obtained at least one usable product includes carbonizing obtained lignocellulose at elevated temperature 600-900° C. under N2 and CO2 to obtained graphite.

5. A method for valorization of risk husks, the method comprising:
providing a quantity of rice husks;
shredding the quantity of rice husks into small husk fragments;
washing the small husk fragments to remove dust and waste from the small husk fragments;
forming a mixture by treating the small husk fragments with an alkaline solution until the pH of the mixture is between 9 and 10, forming a lignin-free cellulose;
forming a silicate-rich filtrate and a silicate-free residue by increasing the pH of the mixture to about 14;
treating the silicate-rich filtrate by using mineral acid to reduce the pH to about 7 to obtain a silicate solution;
reacting the silicate solution with pluronic 127 copolymer under acidic conditions to obtain a homogeneous gel, which by calcination forms a bio-based mesoporous silica;
treating the silicate-free residual with the mineral acid to reduce the pH to about 1 to precipitate lignocellulose;
drying and grinding the lignocellulose into fine powder;
carbonizing the lignocellulose at elevated temperature 600-900° C. under N2 and CO2 to obtained graphite;
converting the graphite into graphene oxide by adding the graphite into concentrated H2SO4 and NaNO3 at 0° C.;
adding stochiometric KMnO4;
after dilution, adding H2O2 to obtain graphene oxide;
washing the silicate-free residue to remove alkaline contaminations and the silicate-free pulp.
carbonizing the silicate-free pulp at elevated temperature 600-900° C. to obtain activated carbon;
functionalizing the bio-based mesoporous silica by doping with nanoparticles of silver wherein a produced material exhibits high cytotoxic activity against three different tumor cell lines of breast (MCF-7), liver (HepG2), and colon (HCT 116) over a concentration range of 0.01 to 1000 g through apoptotic mechanism;
functionalizing the bio-based mesoporous silica by adding amino groups on the surface wherein the obtained material is exhibited high cytotoxic activity against three different tumor cell lines of breast (MCF-7), liver (HepG2), and colon (HCT 116) over a concentration range of 0.01 to 1000 g through apoptotic mechanism; and
fermenting the lignin-free cellulose by Saccharomyces cerevisiae wherein a bioethanol yield produced from the fermentation is 0.454 ml ethanol/g fermentable.

6. The method according to claim 1 wherein the graphite and the activated carbon exhibits high adsorption behavior for the different contaminants present in water such as anionic and cationic dyes, and phenolic compounds.

7. The method according to claim 1 wherein the silver doped bio-based silica is impregnated with lithium ions for investigation as an anti-cancer agent against the cell lines of breast (MCF-7), liver (HepG2), and colon (HCT 116) over a concentration range of 0.01 to 1000 g and wherein the impregnated material exhibited high cytotoxic activity against the three cell lines in an apoptotic mechanism.

8. The method according to claim 1 including shredding raw rice straw into 2-cm pieces using plastic scissors for extraction of silica from the rice husk and straw by using potassium hydroxide.

9. The method according to claim 1 including stirring the rice husk and the shredded rice straw separately in 5% KOH solution at 1:12 (g/ml) weight ratio of solid:solution, respectively, heating to boiling for 30 min, leaving overnight, filtering, and washing twice with distilled water until filtrates becomes neutral.

10. The method according to claim 5 including collecting the filtrates wherein the filtrates contain leached silica, the major component of ash.

11. The method according to claim 6 including adding 10% HCl until the pH of the solutions reached 5-7 to precipitate dissolved silica.

12. The method according to claim 7 including filtering, drying and weighing the precipitated silica.

13. The method according to claim 7 wherein portions of the obtained silica are burnt in an electric muffle furnace at 500, 700, and 900° C. for 1.5 hours.

14. The method according to claim 9 including functionalizing the silica for water desalination studies.

15. A method for valorization of risk husks, the method comprising:
providing a quantity of rice husks;
shredding the quantity of rice husks into small husk fragments;
washing the small husk fragments to remove dust and waste from the small husk fragments;
forming a mixture by treating the small husk fragments with an alkaline solution until the pH of the mixture is between 9 and 10, forming a lignin-free cellulose;
forming a silicate-rich filtrate and a silicate-free residue by increasing the pH of the mixture to about 14;
treating the silicate-rich filtrate by using mineral acid to reduce the pH to about 7 to obtain a silicate solution;
reacting the silicate solution with pluronic 127 copolymer under acidic conditions to obtain a homogeneous gel, which by calcination forms a bio-based mesoporous silica;
treating the silicate-free residual with the mineral acid to reduce the pH to about 1 to precipitate lignocellulose;
drying and grinding the lignocellulose into fine powder;
carbonizing the lignocellulose at elevated temperature 600-900° C. under N2 and CO2 to obtained graphite;
converting the graphite into graphene oxide by adding the graphite into concentrated H2SO4 and NaNO3 at 0° C.;
adding stochiometric KMnO4;
after dilution, adding H2O2 to obtain graphene oxide;
washing the silicate-free residue to remove alkaline contaminations and the silicate-free pulp.
carbonizing the silicate-free pulp at elevated temperature 600-900° C. to obtain activated carbon;
functionalizing the bio-based mesoporous silica by doping with nanoparticles of silver wherein a produced material exhibits high cytotoxic activity against three different tumor cell lines of breast (MCF-7), liver (HepG2), and colon (HCT 116) over a concentration range of 0.01 to 1000 g through apoptotic mechanism;
functionalizing the bio-based mesoporous silica by adding amino groups on the surface wherein the obtained material is exhibited high cytotoxic activity against three different tumor cell lines of breast (MCF-7), liver (HepG2), and colon (HCT 116) over a concentration range of 0.01 to 1000 g through apoptotic mechanism; and
fermenting the lignin-free cellulose by Saccharomyces cerevisiae wherein a bioethanol yield produced from the fermentation is 0.454 ml ethanol/g fermentable.
